(19) United States
(12) Patent Application Publication
(10) Pub. No.: US 2018/0228358 A1
(43) Pub. Date: Aug. 16, 2018

(54) Headset Otoscope

(71) Applicants: David R. Hall, Provo, UT (US); Ben Swenson, Lehi, UT (US); Dan Allen, Springville, UT (US); Justin Robinson, Provo, UT (US)

(72) Inventors: David R. Hall, Provo, UT (US); Ben Swenson, Lehi, UT (US); Dan Allen, Springville, UT (US); Justin Robinson, Provo, UT (US)

(21) Appl. No.: 15/433,137

(22) Filed: Feb. 15, 2017

(51) Int. Cl. A61B 1/227; A61B 1/04; A61B 1/06

(52) U.S. Cl. CPC A61B 1/227; H04R 1/028; A61B 1/06; A61B 1/04

(57) ABSTRACT

An otoscope apparatus is disclosed which, in general, includes a left-ear housing and a right-ear housing connected together by a tensioning band. The left-ear housing and right ear housing each include a light source and a camera. The left-ear and right-ear housings each include a speculum which corresponds to a user's left and right ear canals, respectively.

### BACKGROUND

### Field of the Invention

This invention relates generally to the field of medical otoscopes, and more specifically to headset otoscopes.

### SUMMARY OF THE INVENTION

An invention has been developed in response to present state of the art and, in particular, in response to problems and needs in the art that have not yet been fully solved by currently available systems and methods. Accordingly, a headset otoscope has been developed. Features and advantages of different embodiments of the invention will become more fully apparent from the following description and appended claims, or may be learned by practice of the invention as set forth hereinafter.

An otoscope apparatus is disclosed which, in general, includes a left-ear housing and a right-ear housing connected together by a tensioning band. The left-ear housing and right ear housing each include a light source and a camera. The left-ear and right-ear housings each include a speculum which corresponds to a user's left and right ear canals, respectively.

Each speculum may be attachable to and detachable from the left-ear and right-ear housings, respectively. Additionally, each speculum may have a shape which conforms to a portion of the user's left and right ear canals, respectively.

The otoscope may also include a controller. The controller may be electrically connected to each light source and each camera. The otoscope may further include a wireless transceiver. The wireless transceiver may be electrically connected to the controller. The otoscope may also include a wired communications port. The wired communications port may be electrically connected to the controller.

The otoscope may include a speaker. The otoscope may include a pump. The pump may be fluidly coupled to each speculum. Each speculum may include elastomeric material. Also, each speculum may include a pressure sensor. Each speculum may include a temperature sensor. The otoscope may include a light transceiver. The tensioning band may include an adjustment portion which has adjustable length.

Each speculum may include a lens. Each lens may be a magnifying lens, and each magnifying lens may be a Fresnel lens. Each speculum may include an optical fiber cable. Each speculum may include an acoustic transducer. Each speculum may include a force sensor. The left-ear housing and right-ear housing may each include a cushion.

### BRIEF DESCRIPTION OF THE DRAWINGS

A more particular description of the invention briefly described above is made below by reference to specific embodiments. Several embodiments are depicted in drawings included with this application, in which:

FIG. 1 depicts a headset otoscope apparatus;

FIG. 2 depicts an otoscope apparatus and a user;

FIG. 3 depicts an embodiment similar to FIG. 1 with a controller;

FIG. 4 depicts an embodiment similar to FIG. 3 with a wired communications port;

FIG. 5 depicts an embodiment similar to FIG. 1 with a pump;

FIG. 6 depicts an embodiment similar to FIG. 1 with temperature sensors;

FIG. 7 depicts an embodiment similar to FIG. 1 with a light transceiver;

FIG. 8 depicts an otoscope having an adjustable tensioning band;

FIG. 9 depicts a speculum of an otoscope;

FIG. 10 depicts an embodiment similar to FIG. 1 with an optical fiber cable.

### DETAILED DESCRIPTION

A detailed description of the claimed invention is provided below by example, with reference to embodiments in the appended figures. Those of skill in the art will recognize that the components of the invention as described by example in the figures below could be arranged and designed in a wide variety of different configurations. Thus, the detailed description of the embodiments in the figures is merely representative of embodiments of the invention, and is not intended to limit the scope of the invention as claimed.

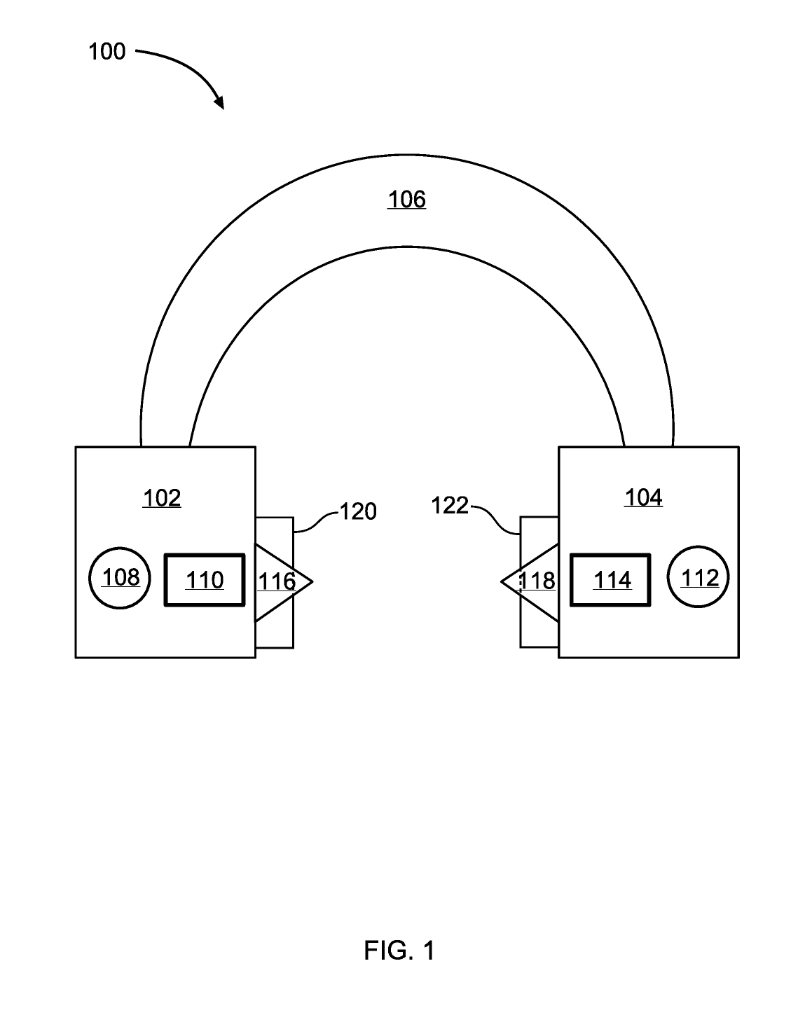

FIG. 1 depicts a headset otoscope apparatus. Otoscope apparatus 100 includes left-ear housing 102 and right-ear housing 104. Left-ear housing 102 and right-ear housing 104 are connected together by tensioning band 106. Left-ear housing 102 includes light source 108 and camera 110. Right-ear housing 104 includes light source 112 and camera 114. Left-ear housing 102 includes speculum 116 which corresponds to a user's left ear canal. Right-ear housing 104 includes speculum 118 which corresponds to the user's right ear canal.

A user may place left-ear speculum 116 into his or her left ear canal and right-ear speculum 118 into his or her right ear canal. Light source 108 and light source 112 may illuminate the user's left and right ear canals, respectively. Camera 110 and camera 114 may subsequently capture images of the user's left and right ear canals, respectively. Camera 110 and camera 114 may each store the captured images in internal memory included in each. Camera 110 and camera 114 may each transfer the captured images wirelessly or via a wire to a peripheral device such as a smartphone, a laptop, a tablet, or a server.

Tensioning band 106 may bias left-ear housing 102 and right-ear housing 104 toward each other causing a resultant force acting on speculum 116 and a user's left ear and causing another resultant force acting on speculum 118 and a user's right ear. Left-ear housing 102 and right-ear housing 104 may include cushion 120 and cushion 122, respectively. Cushion 120 and cushion 122 may protect the user's respective ears from resultant forces caused by tensioning band 106. Tensioning band 106 may be made of any of a variety of materials, including metal, polymer, elastomer, and composite materials.

Light sources 108 and 112 may include any of a variety of light emitters, including light emitting diodes (LEDs), incandescent light bulbs, and fluorescent light bulbs. Left-ear housing 102 and right-ear housing 104 may passively direct light toward openings in speculum 116 and speculum 118, respectively. Left-ear housing 102 and right-ear housing 104 may include reflective inner surfaces which may redirect light from light source 108 and light source 112, respectively. Camera 110 and camera 114 may include film. Camera 110 and camera 114 may include any of a variety of image sensors, including charge-coupled device (CCD) sensors or complementary metal-oxide-semiconductor (CMOS) sensors.

Speculum 116 and speculum 118 may be made of any of a variety of materials, such as metal, polymer, elastomer, composite, ceramic, or acrylic materials.

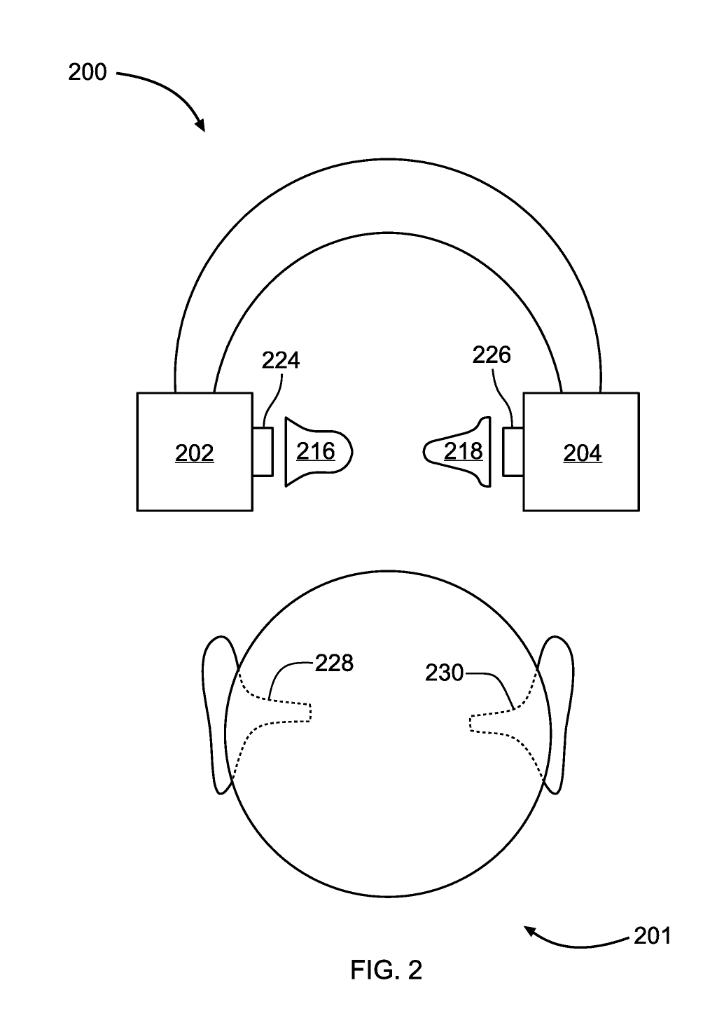

FIG. 2 depicts an otoscope apparatus and a user. Otoscope apparatus 200 may include left-ear housing 202 and right-ear housing 204. Left-ear housing 202 may include attachment knob 224, and right-ear housing 204 may include attachment knob 226. Knob 224 may be permanently affixed to an outer surface of left-ear housing 202. Knob 226 may be permanently affixed to an outer surface of right-ear housing 204. Speculum 216 may be attachable to and detachable from left-ear housing 202 via knob 224. Speculum 218 may be attachable to and detachable from right-ear housing 204 via knob 226. User 201 may have left-ear canal 228 and right-ear canal 230. Speculum 216 and speculum 218 may each have a shape which conforms to a portion of left-ear canal 228 and right-ear canal 230, respectively.

Speculum 216 and speculum 218 may be manufactured to fit left-ear canal 228 and right-ear canal 230, respectively. Speculum 216 and speculum 218 may include a material such as an elastomer or foam which may conform to geometry of left-ear canal 228 and right-ear canal 230.

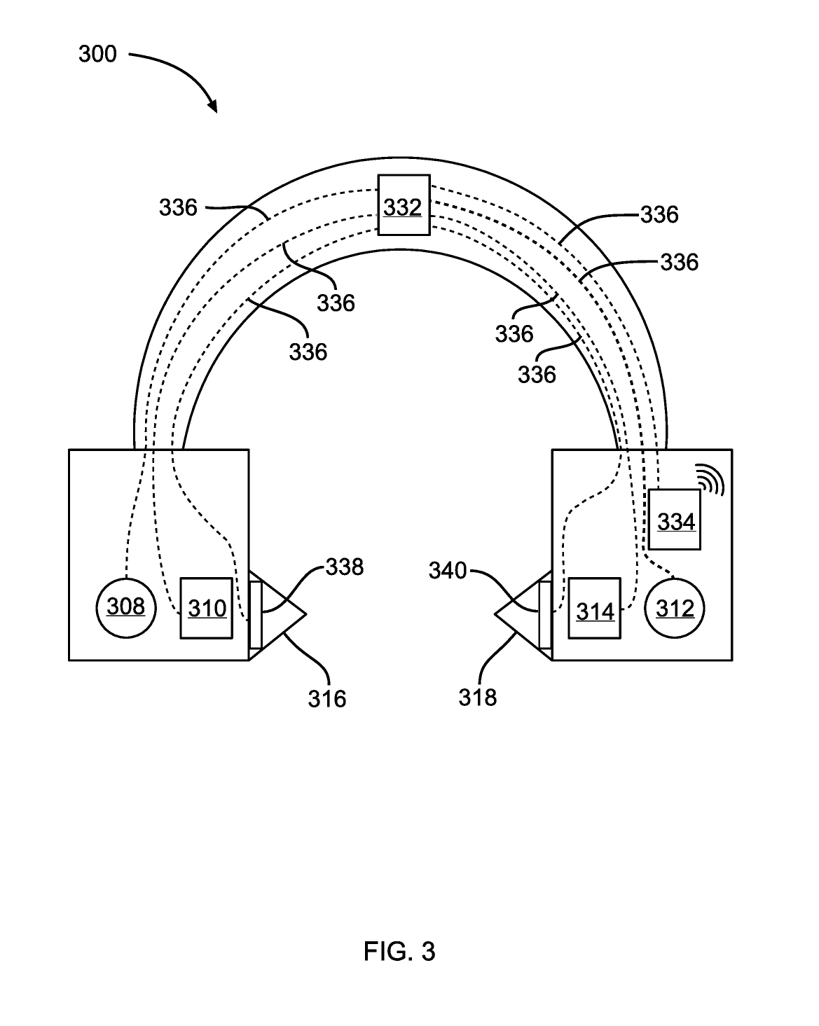

FIG. 3 depicts an embodiment similar to FIG. 1 with a controller. Otoscope apparatus 300 may include controller 332. Controller 332 may be electrically connected to light source 308 and light source 312 via wires 336. Controller 332 may also be electrically connected to camera 310 and camera 314 via wires 336. Otoscope apparatus 300 may also include wireless transceiver 334. Wireless transceiver 334 may be electrically connected to controller 332 via wires 336. Controller 332 may be powered by any of a variety of means, including via a battery, wirelessly, or via a power outlet. When left-ear speculum 316 and right-ear speculum 318 are placed into a user's left and right ear canals, respectively, controller 332 may actuate light source 308 and light source 312. Controller 332 may also actuate camera 310 and camera 314. Camera 310 and camera 314 may send image data to controller 332, and controller 332 may subsequently send the image data via wireless transceiver 334 to a peripheral device or server. The peripheral device, such as a smartphone or laptop computer, may display the image data as it is received and/or may store the image data for later use.

Left-ear speculum 316 may include force sensor 338. Right-ear speculum 318 may include force sensor 340. Force sensor 338 and force sensor 340 may be electrically connected to controller 332 via wires 336. Controller 332 may actuate light source 308 and light source 312 in response to signals sent from force sensor 338 and force sensor 340. Force sensor 338 and force sensor 340 may collect data, and may send the data to controller 332. Controller 332 may subsequently send the data via wireless transceiver 334 to a peripheral device or server.

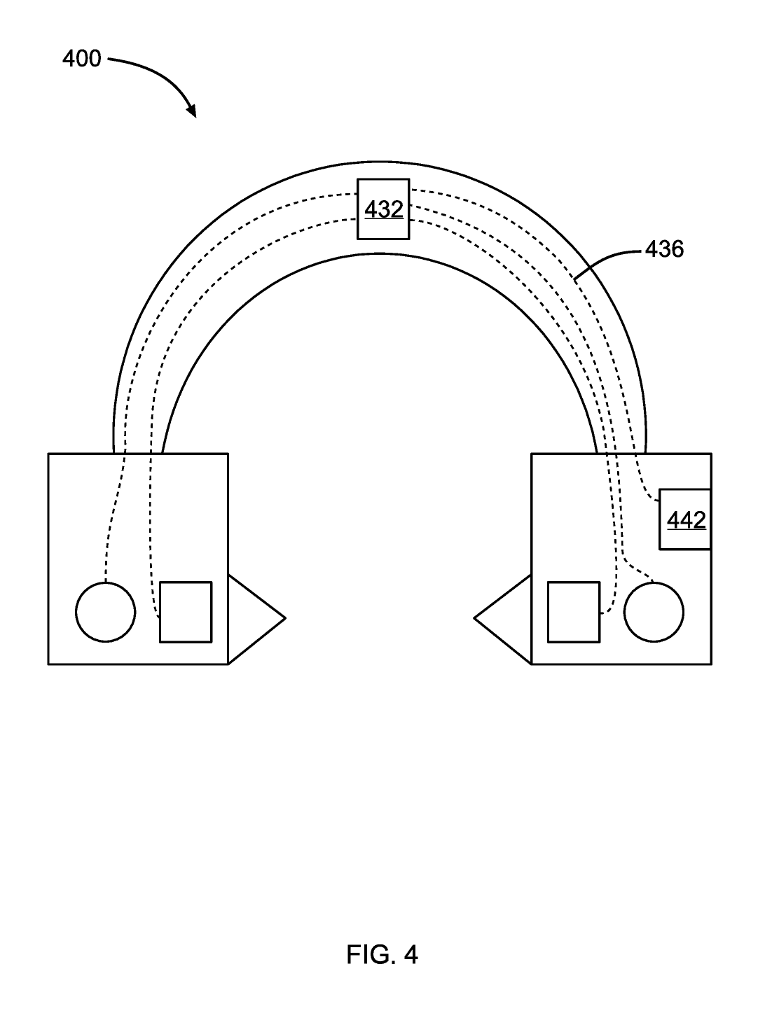

FIG. 4 depicts an embodiment similar to FIG. 3 with a wired communications port. Otoscope apparatus 400 may include wired communications port 442. Wired communications port 442 may be electrically connected to controller 432 via wires 436. Controller 432 may send data via wired communications port 442 to a peripheral device or server. Wired communications port 442 may connect to a peripheral device or server by any of a variety of means, including via a universal serial bus (USB) cable, a high-definition multimedia interface (HDMI) cable, or a digital visual interface (DVI) cable.

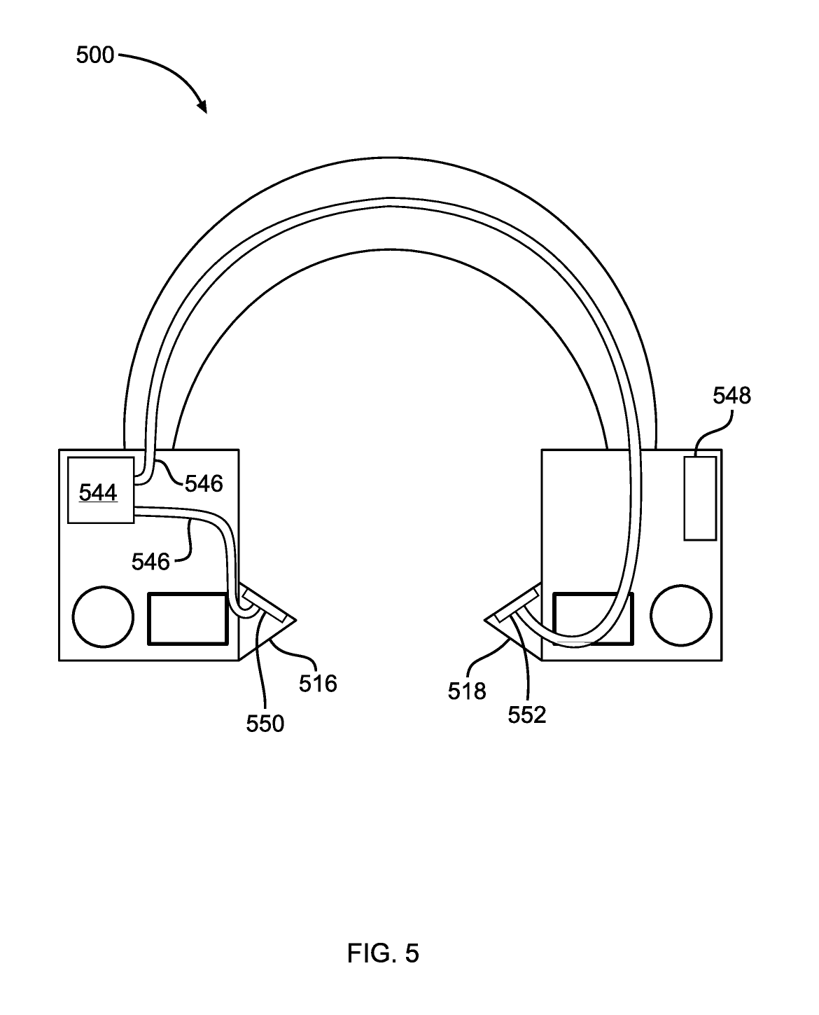

FIG. 5 depicts an embodiment similar to FIG. 1 with a pump. Otoscope apparatus 500 may include pump 544. Pump 544 may be fluidly coupled to left-ear speculum 516 and right-ear speculum 518. Speculum 516 and speculum 518 may each include openings. Speculum 516 and speculum 518 are placed into a user's left-ear canal and right-ear canal, respectively. Pump 544 may be actuated, wherein pump 544 may push air into a user's left-ear canal and right-ear canal through openings in speculum 516 and speculum 518, respectively.

Speculum 516 and speculum 518 may include a foam or elastomeric material. The foam or elastomeric material may change shape due to a pressure caused by pump 544 such that speculum 516 and speculum 518 may conform to geometry of a user's left-ear canal and right-ear canal, respectively. Otoscope apparatus 500 may include speaker 548. Speaker 548 may relay instructions to a user, such as directions for when to remove speculum 516 and speculum 518. Speculum 516 may include pressure sensor 550 and speculum 518 may include pressure sensor 552. Pressure sensor 550 and pressure sensor 552 may collect pressure data, and may relay pressure data back to pump 544 such that pump 544 will stop or start at preset values.

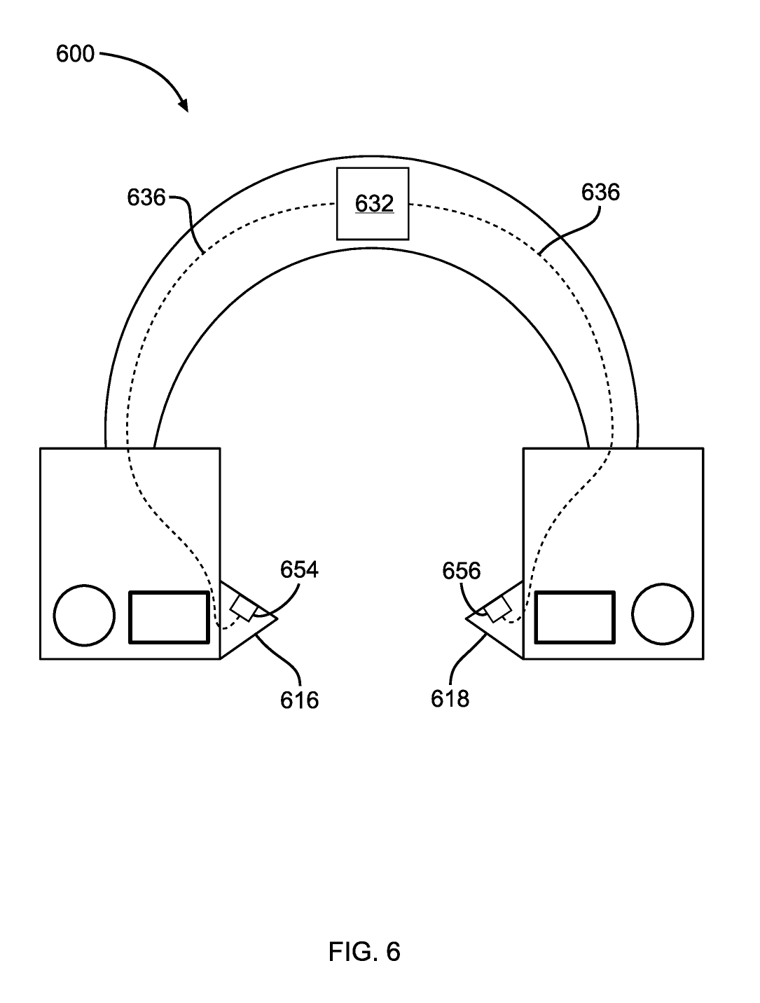

FIG. 6 depicts an embodiment similar to FIG. 1 with temperature sensors. Left-ear speculum 616 may include temperature sensor 654 and right-ear speculum 618 may include temperature sensor 656. Temperature sensor 654 and temperature sensor 656 may be electrically connected to controller 632 via wires 636. When speculum 616 and speculum 618 are placed into a user's left and right ear canals, respectively, temperature sensors 654 and 656 may collect temperature data and send the temperature data to controller 632. The temperature data may be used to determine internal body temperature of the user.

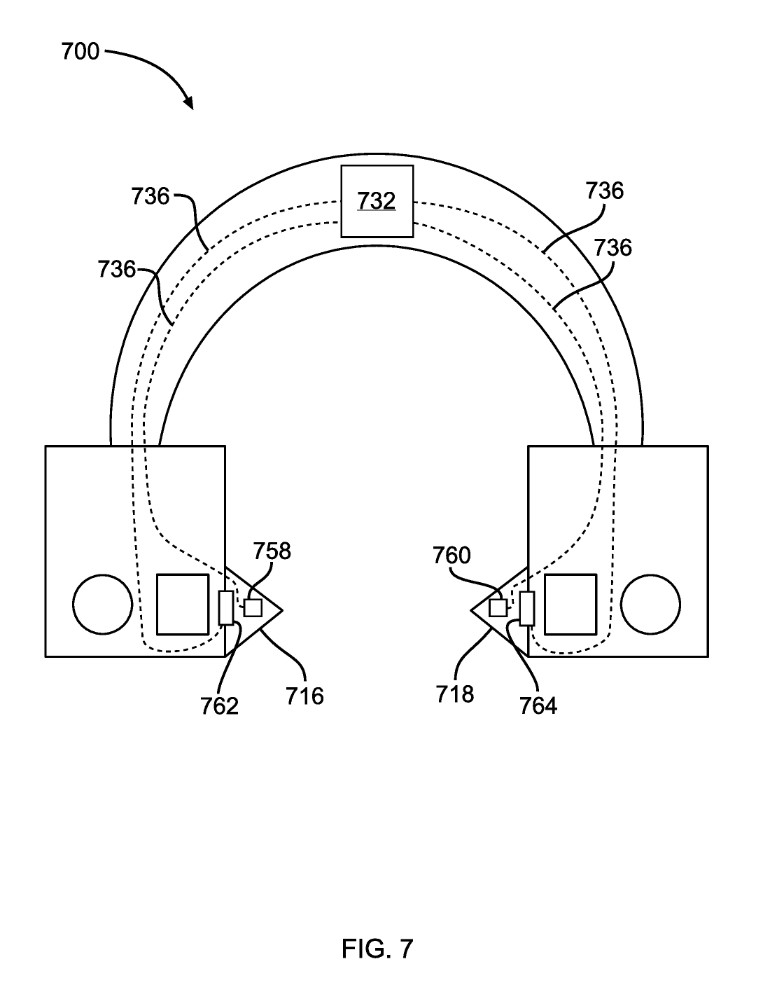

FIG. 7 depicts an embodiment similar to FIG. 1 with a light transceiver. Otoscope apparatus 700 may include light transceiver 758 positioned within left-ear speculum 716. Otoscope apparatus 700 may also include light transceiver 760 positioned within right-ear speculum 718. Light transceiver 758 and light transceiver 760 may transmit and receive light having wavelengths of magnitude less than or equal to those of infrared light. Light transceiver 758 and light transceiver 760 may send data to controller 732 via wires 736 for storage and processing.

Light transceiver 758 and light transceiver 760 may utilize photoplethysmography to determine pulse rate or pulse wave velocity of a user (where pulse wave velocity is a measure of arterial stiffness). Photoplethysmography is a technique wherein a light transceiver illuminates skin of a person and measures changes in light absorption. The light absorption of the tissue changes with perfusion of blood to dermis and subcutaneous tissue. This perfusion occurs over time as a function of the user's heartbeat.

Speculum 716 may include acoustic transducer 762, and speculum 718 may include acoustic transducer 764. Acoustic transducer 762 and acoustic transducer 764 may emit sound waves into a left-ear canal and a right-ear canal of a user. Tissue and bone of a user may reflect the sound waves, and acoustic transducer 762 and acoustic transducer 764 may receive the reflected sound wave data. Acoustic transducer 762 and acoustic transducer 764 may subsequently send the sound wave data to controller 732 via wires 736. Controller 732 may store the sound wave data and may send the sound wave data to a peripheral device.

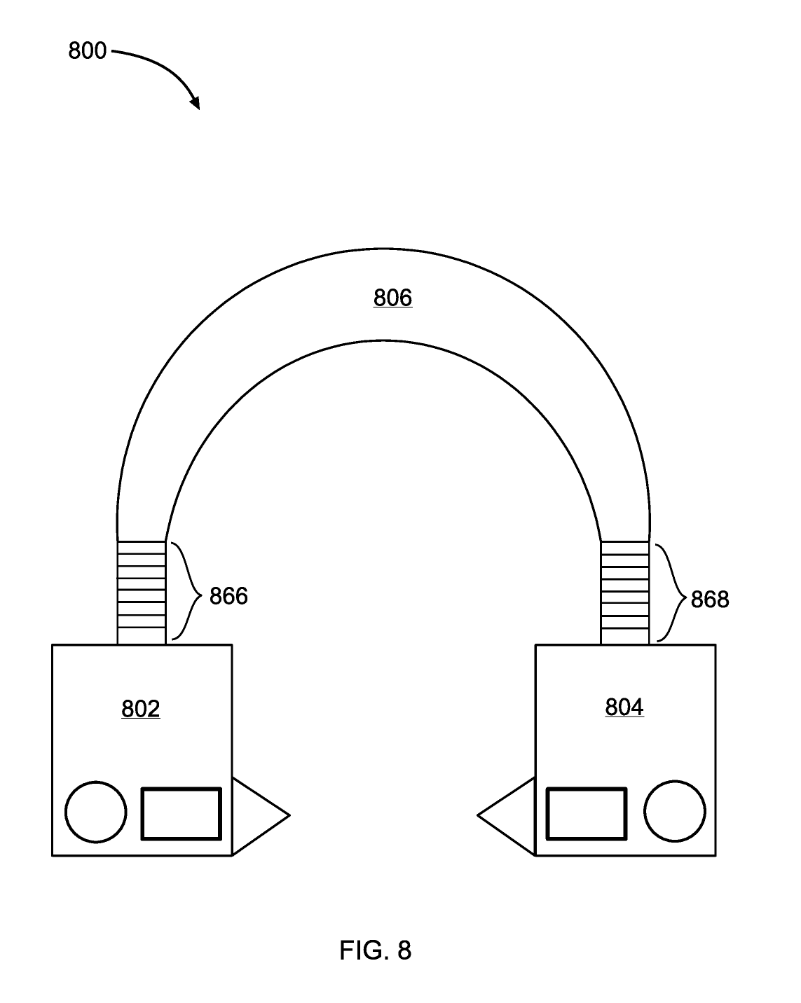

FIG. 8 depicts an otoscope having an adjustable tensioning band. Otoscope apparatus 800 may include left-ear housing 802 and right-ear housing 804 connected together by tensioning band 806. Tensioning band 806 may include adjustment portion 866 and adjustment portion 868, corresponding to housing 802 and housing 804, respectively. Adjustment portion 866 and adjustment portion 868 may have adjustable length. A user may adjust length of adjustment portion 866 and adjustment portion 868 such that housing 802 and housing 804 may move relatively closer or further away with respect to tensioning band 806. Extending or contracting adjustment portion 866 and adjustment portion 868 may allow the user to fit otoscope apparatus 800 around his or her head.

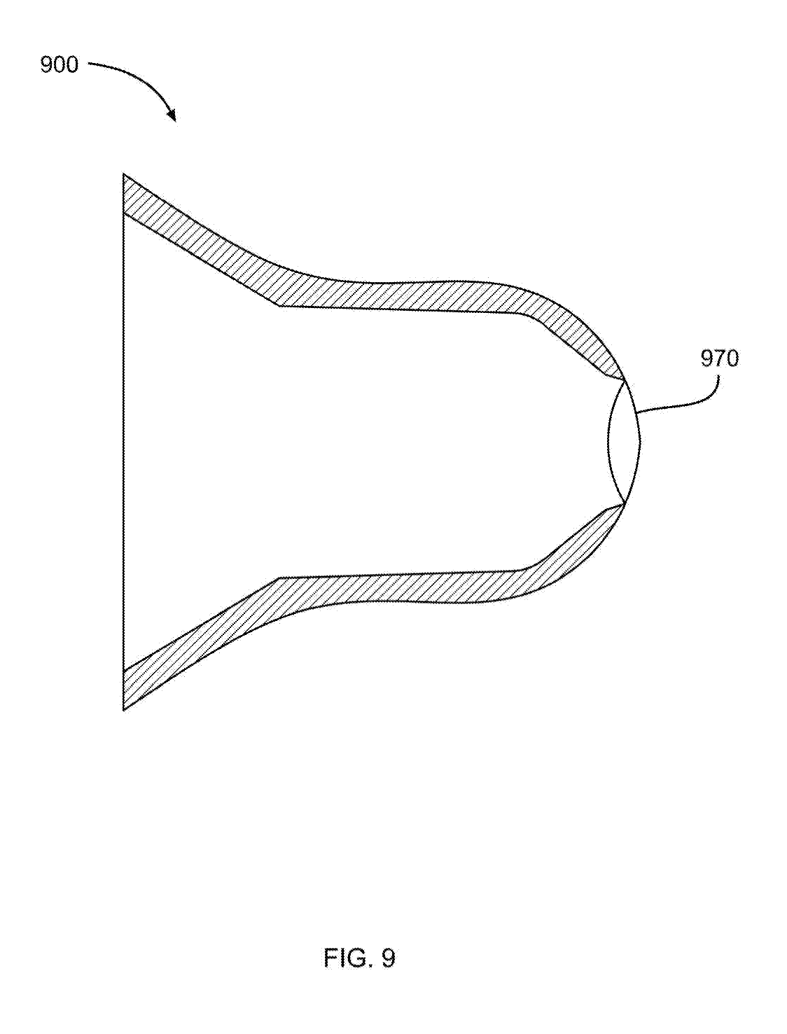

FIG. 9 depicts a speculum of an otoscope. Speculum 900 may include lens 970. Lens 970 may passively transmit light to and from speculum 900. Lens 970 may be a convergent or a divergent lens. Lens 970 may be a magnifying lens, which may allow greater magnification of a view of a user's ear canal. Lens 970 may be Fresnel lens, which may allow for greater compactness of lens 970.

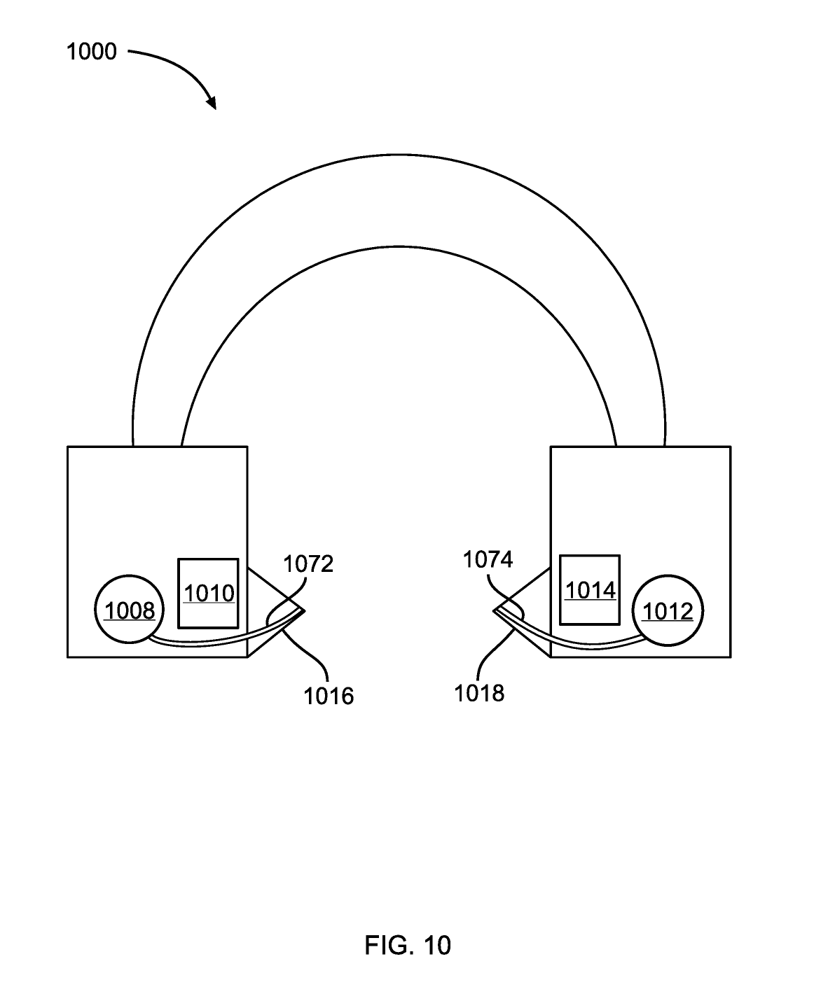

FIG. 10 depicts an embodiment similar to FIG. 1 with an optical fiber cable. Left-ear speculum 1016 of otoscope apparatus 1000 may include optical fiber cable 1072. Right-ear speculum 1018 may include optical fiber cable 1074. Optical fiber cable 1072 and optical fiber cable 1074 may be optically coupled to light source 1008 and light source 1012, respectively.

Optical fiber cable 1072 and optical fiber cable 1074 may transmit light from light source 1008 and light source 1012, respectively, to corresponding ear canals of a user. Optical fiber cable 1072 and optical fiber cable 1074 may be positioned along walls within speculum 1016 and speculum 1018 in order to provide an unobstructed view for camera 1010 and camera 1014, respectively. Light may subsequently be collected from corresponding ear canals of the user to camera 1010 and camera 1014, respectively. Speculum 1016 and speculum 1018 may each have an orifice or lens through which optical fiber cables 1072 and 1074 may transmit light, and/or through which light may be transmitted back to cameras 1010 and 1014, respectively.

1. An otoscope apparatus comprising:
a left-ear housing and a right-ear housing connected together by a tensioning band;
the left-ear housing and the right-ear housing each comprising a light source and a camera; and
the left-ear and right-ear housings each further comprising a speculum corresponding to a user's left and right ear canals, respectively.

2. The otoscope apparatus of claim 1, wherein each speculum is attachable to and detachable from the left-ear and right-ear housings, respectively.

3. The otoscope apparatus of claim 2, wherein each speculum has a shape which conforms to a portion of the user's left and right ear canals, respectively.

4. The otoscope apparatus of claim 1, wherein the left-ear housing and the right-ear housing each comprise a cushion.

5. The otoscope apparatus of claim 1, further comprising a controller electrically connected to each light source and each camera.

6. The otoscope apparatus of claim 5, further comprising a wireless transceiver electrically connected to the controller.

7. The otoscope apparatus of claim 5, further comprising a wired communications port electrically connected to the controller.

8. The otoscope apparatus of claim 5, wherein each speculum comprises a force sensor electrically connected to the controller

9. The otoscope apparatus of claim 1, further comprising a speaker.

10. The otoscope apparatus of claim 1, further comprising a pump which is fluidly coupled to each speculum.

11. The otoscope apparatus of claim 10, wherein each speculum comprises an elastomeric material.

12. The otoscope apparatus of claim 11, wherein each speculum further comprises a pressure sensor.

13. The otoscope apparatus of claim 1, wherein each speculum comprises a temperature sensor.

14. The otoscope apparatus of claim 1, further comprising a light transceiver.

15. The otoscope apparatus of claim 1, wherein each speculum comprises an acoustic transducer.

16. The otoscope apparatus of claim 1, wherein the tensioning band comprises an adjustment portion which has adjustable length.

17. The otoscope apparatus of claim 1, wherein each speculum comprises a lens.

18. The otoscope apparatus of claim 17, wherein each lens is a magnifying lens.

19. The otoscope apparatus of claim 18, wherein each lens is a Fresnel lens.

20. The otoscope apparatus of claim 1, wherein each speculum comprises an optical fiber cable.
